(19) United States
(12) Patent Application Publication
(10) Pub. No.: US 2005/0136024 A1
(43) Pub. Date: Jun. 23, 2005

(54) Dermatological compositions

(76) Inventor: Richard F. Stockel, Bridgewater, NJ (US)

Correspondence Address: RICHARD F. STOCKEL, 475 ROLLING HILLS ROAD, BRIDGE WATER, NJ, 08807, US

(21) Appl. No.: 10/741,346

(22) Filed: Dec. 22, 2003

(51) Int. Cl. A61K 7/06; A61K 7/11; A61K 31/785

(52) U.S. Cl. 424/70.17; 424/78.27

(57) ABSTRACT

This invention discloses new and novel dermatological compositions synthesized by a metathesis or an acid-base reaction, where both reactants have bioactivity, resulting in improved properties for the treatment of various skin conditions. The novel compositions are preventive as well as therapeutic in alleviating the symptoms of skin disorders associated with disturbed keratinzation or inflammation.

### BACKGROUND OF THE INVENTION

1. Field of the Invention

This invention relates to the treatment of skin disorders, and to a new generic approach in combining and forming new compositions of existing dermatological active compounds. This development has lead to a broad range of compounds having exceptional antimicrobial activity with associated benefit to a host of skin conditions including, but not limited to ichthyosis, eczema, dry skin, psoriasis, pruritus, palmar, plantar hyperkeratosis, acne, keratoses, herpes virus infection.

The present invention provides compositions and methods for alleviation of both visible and non-visible, or preemergent, dermatological lesions associated with changes in normal keratinzation, cutaneous infection, epidermal formation or pilosebaceous function, such as acne, psoriasis, seborrhea, ingrown hairs and pseudofolliculitis barbae, and hyper-pigmented skin. The invention compositions comprise dermatologically topical antimicrobial, antibiotic, antibacterial, antifungal agents, or combinations thereof.

Further, the invention relates to dermatological agents with reduce irritation caused frequently by many of the topical compounds presently being used today.

This invention greatly expands the treatment of skin diseases and cosmetic conditions by allowing the medical profession to choose from a plethora of bioactive agents having the proper chemical characteristics needed to prepare the resulting compositions for a specific skin problem. For example anti-fungal, antibiotic, and antimicrobial cationic (or conjugate base) can be combined with anionic (or conjugate acid) to give the desired therapeutic outcome.

Benzoyl peroxide is frequently found in anti-acne preparations in combination with antibacterial or antibiotics to effectively extend its spectrum of activity, e.g., U.S. Pat. No. 5,767,098. However, there are major drawbacks with this combination approach. With prolonged usage the bacterial flora become resistant, rendering the antibiotics less effective in subsequent treatment. Furthermore, the benzoyl peroxide component tends to be oxidatively unstable. The compositions of this invention do not have these short-comings as a skin caring product.

2. Prior Art

Numerous patents have disclosed the use of alpha hydroxy acids for the treatment of skin conditions. U.S. Pat. No. 4,363,815 describes the use of these compounds for the treatment of dry skin, ichthyosis, plantar hyperkeratosis, Darier's disease, keratoses, acne, psoriasis eczema, pruritus, warts and herpes. Other patents describe the use of various incipients to lessen irritation and stinging like lactate salts the use of amphoteric salt (U.S. Pat. No. 5,420,106), the use of ascorbic acid derivatives (U.S. Pat. No. 5,703,122) the use of amino acids disclosed in Cosmetics and Toiletries, V113, March, 1998, p55.

Salicylic acid is frequently mentioned as an active ingredient to treat a whole host of skin conditions like psoriasis, skin atrophy, skin wrinkles, acne an other skin problems as disclosed in U.S. Pat. Nos. 5,776,920; 5,780,457; 5,780,458; 6,436,417. In all these cases salicylic acid must be modified with other incipients to prevent side-reactions like irritation and the like. The compositions of this invention can be synthesized directly not having this undesirable chemical property by pre-selection of the bioactive cationic and anionic components.

There is a need for the treatment of severe acne, since the only treatment, which has been proven to be uniformly effective in the oral administration of isotretinoin. This medication has many side effects with the potential to cause birth defects as the most serious one.

Therefore, there is a pressing need for new and more efficacious medicines useful in the broad spectra of skin conditions that exist, which minimize side-reactions and effectively treats the specific condition at hand.

### INVENTION

This invention relates to new biocidal complexes prepared by metathesis synthesis or by an acid-base reaction. In practice the metathesis reaction is carried out by reacting a bioactive cationic species, which can be either monomeric or polymeric, with a bioactive anion, which can also be monomeric or polymeric, in a suitable solvent system, whereby the complex is precipitated from solution.

Another method to prepare some of the complexes of this invention is by an acid-base reaction, whereby the bioactive acid can donate a proton to the bioactive base. Generally, these reactions involve Bronsted acid-base species.

These complexes tend to have low water solubility; therefore they must be applied as emulsions, nano-emulsions, microemulsions, gels, dispersions, or creams.

The complexes could also be applied orally, and therefore would need to be formulated in a tablet form as is well known in the pharmaceutical field by anyone skilled in the field.

Individually, the skin bioactives of this invention are well known in the published literature. In fact the literature is replete with examples of using admixtures of two or more bioactives (non-complexes), e.g., U.S. Pat. No. 5,505,949 discloses the admixture of clotrimazole (antifungal), betamethasone dispropionate (steroid), and salicylic acid (antiseptic-antibacterial-kerololytic) into a topical cream. There are definite limits to this approach such as compatibility, long-term stability and the uneven adsorption of each component into the skin structure for therapeutic maximum value. A singular composition like the complexes of this invention would solve all of these problems.

In accordance with this invention, the effectiveness of individual biologically active compounds can be enhanced by the formation of these complexes as described by this invention. Thus the combination of a bioactive cation with a bioactive anion improves the overall biological activity for the treatment of various skin conditions.

This invention has important safety and toxicity implications because most of the reactants are well known in the literature and have undergone numerous testing for either EPA and/or FDA approval.

Another advantage involves the green chemistry used in the synthesizing these compositions. The metathesis reaction can be conducted in a totally aqueous medium. The by-product of this reaction is a salt, which does not represent any serious environmental problem for disposal. In fact, many salts can be recycled for other uses.

The acid-base reactions usually involve aqueous, aqueous-alcohol, or alcohol as solvents. In all cases the solvents can be recycled by distillation.

The following monomeric and polymeric cationic anionic, acids, and bases are illustrative of this invention. They by no means represent all possibilities, but instead are examples of the broad array available to a practitioner who wishes to carry out the scope of this invention.

List of Some General Bioactive Cationic Classes and Some Specific Examples for Each

Polymeric Biocides

Polybiguanides—Polyhexamethylene biguanide HC1 salt
Polyguanidines—polyhexamethylene guanidine HC1 salt
Polyionenes—poly (oxyethylene (dimethylimino) ethylene (dimethylimino) ethylene dichloride
Polyaminosaccharides—Chitosan salts
Quaternary ammonium dendrimeric biocides as described in U.S. Pat. No. 6,440,405
   Monomeric Biocides
Quats—benzalkonium chloride, didecyldimethyl ammonium chloride, cetyl pyridinium chloride
Biguanides—alexidine, Hexetidine
Amidine—Propamidine, Dibromopropamidine
Gemine quats—ethanediyl—α, w-bis (dodecyldimethyl) ammonium halide
   Amine Antibiotic Acid Salts
Tetracycline HC1 salt
Clindamycin HC1 salt
Tazarotene HCl salt
Erythromycin HCl salt
Clinafloxacin HCl salt
Doxycycline HC1 salt
Minocycline HC1 salt
Lincomycin
   Azole Antifungal Salts
Cloconazole
Clotrimazole
Cyproconazole
Fenbuconazole
Myclobutanil
Propiconazole
Tebuconazole
Triadimefon
Miconazole
Flucytosine
   List of Some General Bioactive Anionic Classes and Some Specific Examples for Each

Listed in the non-disassociated form
phenolics—triclosan, hexyresorcinol, thymol
Hydroxy carboxylic acids—lactic, glycolic, gluconic, glyceric, salicylic
Beta keto carboxylic acids—acetopyruvic
Carboxylic acids—undecylenic, pantothenic, azelaic, all trans retenoic, tretinoin, isotretinoin, adapalene,
Sulfonamide—sulfacetamide
   List of Some General Bioactive Carboxylic Acids and Some Specific Examples of Each, Capable of Reacting with Basic Amine Compounds
Carboxylic acids—salicylic, hydroxy carboxylic, alpha or beta carboxylic, azelaic, clinafloxacin, adapalene, pantothenic, retinoic, and undecylenic, sulfonamide—sulfacetamide
   List of Some Specific Bioactive Basic Amine Compounds Capable of Reacting with Carboxylic Acids of this Invention
Amine containing bases—clotrimazole, clindamycin, erthromycin, tetracycline miconazole, tebuconazole, Chitosan, sulfacetamide, lincomycin, tazarotene, metroridazole, minocycline, and doxycycline.

The compositions (complexes) disclosed in this invention are useful for many known skin conditions as topical administration, or oral applications. These skin problems include dry skin, xerosis, ichthyosis, dandruff, acne, keratoses, psoriasis, wrinkles, anti-aging, warts, blemished skin, hyperpigmented skin, inflammatory dermatoses, eczema, pruritis, hyperkorotic skin, lentigines, melasma, age spots, laxity, leathing texture, roughness, sallow complexion, scaling, telangiectasis, mottled pigmentation, skn atrophy, and skin changes associated with intrinsic aging and photodamage.

Metathesis Procedure

In carrying out the synthesis via the Metathesis reaction the preferred monomeric and polymeric cationic antimicrobial agents are biguanides, guanidines, polyionenes, amidines, quats, dendrimers, amine salts of antibiotics and amine salts of azole antifungal compounds.

With respect to the bioactive anionic portion in the Metathesis reaction the preferred anions are phenols, hydroxy carboxylic acids, beta keto acids, certain carboxylic acids (listed), vitamin A acids and adapalene.

The formation of the complexes are preferably carried out in aqueous solution whenever possible. In order for this reaction to be useful, the resulting complex must precipitate from the solution. In some cases it may be necessary to perform the reactions in organic solvents, nevertheless the starting reactants must be soluble in the solvent(s) and the product must be readily insoluble to achieve good yields. Alcohols, glycols, and glycol ethers are useful solvents to be used in some cases, when necessary.

The reaction takes place at room temperature or evaluated temperatures from 20° to about 90° C., and the reaction is generally completed within one hour.

The final product (complex) is readily removed by decantation of the solvent and the product is dried in a vacuum over at about 75° C. for several hours.

If necessary purification can be readily performed by recrystallization or chromatopgraphic separation.

Acid-Base Procedure

This well known facile reaction can be utilized in some cases by the reaction of a conjugate base (free base) of a biocidal cation with the conjugate acid (protonated) of the biocidal anion. This can be represented by the following example:

| | |
|---|---|
| Chlorhexidine + undecylenic acid → | chlorhexidium |
| Base acid | undecylenate |
| | Complex |

In order for the acid-base synthesis to give good yields, the acid component must have a transferable proton (pka) to a basic molecule (pkb). The reaction is usually conducted in refluxing from 1 to 10 hours in alcohol (C₁-C₄), or aqueous alcoholic solutions. The product is isolated after evaporating off the solvent(s). Recrystallization or chromatographic purification is preferred, if necessary.

It has been found that the acid-base reaction is advantageous but not limiting for the formation of complexes involving amine containing antifungal azoles and antibiotics, when reacting them with antimicrobial agents capable of donating a proton.

Applications of Complexes

The complexes of this invention maybe employed with any of a variety of dermatological or skin care acceptable carriers or excipients normally employed in compositions for topical administration. These are well known to the skilled artisan and include, for example, safe solvents, surfactants, emulsifiers, stabilizers, emollients, humectants, chelating agents, fragrances, skin permeation enhancers, and the like.

The complexes maybe in the form of solutions, emulsions, suspensions, lotions, creams, gels, sticks, ointments, liposomes, aerosol sprays, polymeric gels, plasters, patches, films or tapes, the preparations of which are well known to those skilled in the art of topical pharmaceutical formulations.

As will be recognized by those skilled in the art, the term “effective amount” relates to the conditions under treatment. Some conditions may require treatment with large amounts of the complex. Others may be effectively treated with smaller amounts. The treatment may require one or multiple dosage units applied all at once or a period of time. In any event the skilled artisan will have no difficulty in determining an “effective amount” for the treatment of a specific conditions, by the application of the routine tests procedures normally employed.

Solvents

Since the majority of these complexes of this invention are mostly water tinsoluble (<1 wt %) or only slightly soluble in water, an appropriate solvent is required to solublize it in order to apply the product in the form of a spray, emulsion, nanoemulsion, microemulsion, gel, cream, etc.

Experimentally, it has been found that when the complex has considerable ionic character (a high solubility parameter number), it is incumbent to choose one or more of the following polar solvents, e.g., alcohols (C₁-C₄), glycols, glycol ethers, glycol esters, di, tri and poly hydroxylic liquids, polyglycols (not all inclusive), and the like.

When the complex has a predominance of covalent bonding, them it may be necessary to use less polar or aprotic dipolar solvents in part, or in toto. Examples of these solvents (not all inclusive) are DMF, DMSO, NMP, morpholine N-oxide, dimethyl-2-piperidone, gamma lactone, cyclic amides, C₆-C₁₂ alcohols, mono, di or tri alkyl pkosphates, and the like.

Due to irritation and toxicity considerations the preferred solvents are ethanol, isopropanol, glycerin, propylene glycol, and poly glycols. The latter can be composed of ethylene oxide, propylene oxide, or combinations thereof.

Surfactants

Experimentally, it has been determined that the preferred surfactants, which form microemulsions or emulsions with the compositions of this invention, are by and large, either of the amphoteric non-ionic type, and cationic types or combinations thereof. Highly charged anionic surfactants have the potential to reduce the overall bioactivity of these complexes by causing some degree of precipitation, thereby lessening its effectiveness.

Surfactants that carry a positive charge in strongly acidic media, carry a negative charge in strongly basic media, and form zwitterionic species at intermediate pH's are amphoteric. The preferred pH range for the stability and effectiveness is from about 5.0 to about 9.0. Under this pH range the amphoteric surfactant is mostly or fully in the zwitter (overall neutral charge) form, thereby negating any dilution of bioactivity of the compositions of this invention.

There are several classes of amphoteric surfactants useful for preparing microemulsions or emulsions for the complexes of this invention. These are:
1. N-alkylamino acids
2. alkyldimethyl betaines
3. alkylamino betaines
4. sulfobetaines
5. imidazolines
6. amino or imino propionates

Some of the above amphoteric surfactants have moderate to good antimicrobial activity against certain microorganisms, and hence can be synergistic.

Nonionic surfactants have also been found to be useful to form small particle micelles for these complexes. These can be classified as the following:
1. alcohols
2. alkanolamides
   a. alkanolamides
   b. ethoxylated (propoxylated) amides
3. Amine oxides
4. Esters
   a. ethoxylated (propoxylated) carboxylic acids
   b. ethoxylated (propoxylated) glycerides
   c. glycol esters (and derivatives)
d. mono (di) glycerides
   e. polyglycerol esters
   f. polyhydric alcohol esters and ethers
   g. sorbitan/sorbital esters
   h. di (tri) esters of phosphoric acid
5. Ethers
   a. ethoxylated (propoxylated) alcohols
   b. ethoxylated (propoxylated) lanolin
   c. ethoxylated (propoxylated) polysiloxanes
   d. ethoxylated-propoxylated block copolymers

Suitable cationic surfactants which have been found useful in preparing microemulsions and/or emulsions include D,L-2-pyrrolidone-5-carboxylic acid salt of ethyl-N-cocoyl-L-arginate (CAE), marketed by Ajinomato and cocamidopropyl lauramidopropyl PG dimonium chloride phosphate (PTC) sold by Uniqema.

It has been observed that the choice of a effective surfactant system will differ to some degree for each biocidal complex. The choice will depend on the surfactants hydrophilic-lipophilic balance (HLB) to form a stable small particle micelle in an aqueous or aqueous cosolvent medium solution. Also the combination of two or more amphoteric or a amphoteric-nonionic system or two or more nonionic surfactants or a cationic-amphoteric or cationic-nonionic can also be utilized to achieve satisfactory results.

It has been found that effective concentrations (based on the weight of the complex) of surfactants are in the range of 0.4 weight percent to about 6.0 weight percent.

Other adjivants useful in formulating the complexes of this invention into o/w or w/o type creams, gels and the like are polyether—modified silicone, cyclic silicon, methyl polysilicone, polyoxyethylene castor oil, cetostearyl alcohol, neopentyl glycol dicaprate, sorbitan monosterate, polyvinyl alcohol, propylene glycol, glycerin, Carbowax, glyceryl ether, cholesteryl isostearate, ethanol, isopropanol, glycerol monostearate, PE G100 stearate, hydroxymethyl cellulose, cetyl alcohol, lawryl glucoside, and the like. Other commercial products are available and could be substituted by anyone skilled in the art of formulating dermatological or skin care products.

1. A method for treating dermatological conditions with complexes prepared by a metathesis reaction between bioactive monomeric or polymeric cationic molecule with a bioactive monomeric or polymeric anionic molecule.

2. A method for treating dermatological conditions with complexes prepared by a acid-base reaction between a bioactive organic free base and a bioactive organic molecule capable of donating a proton to the free base.

3. The method as defined in claim 1 wherein the cationic monomer or polymer is used as part of the dermatological complex.

4. The method as defined in claim 3 wherein the cation is a amidine, guanidine biguanide, quaternary, amine acid salts of azoles, amine acid salts of antibiotics, gemini quats, dendrimeric quats, and monomeric or polymeric aminosaccharides acid salts or combinations thereof.

5. The method as defined in claim 4 wherein the cation is a polybiguanide salt or a monomeric biguanide salt.

6. The method as defined in claim 5 wherein the polymer is polyhexamethylene biguanide salt.

7. The method as defined in claim 5 wherein the monomer is a chlorhexidine salt.

8. The method as defined in claim 4 wherein the cation is an amino polysaccharide salt.

9. The method as defined in claim 8 wherein the cation is a chitosan salt.

10. The method as defined in claim 4 wherein the cation is an amine acid salt of a azole composition.

11. The method as defined in claim 10 wherein the cation is a cloconazole, clotrimazole cyproconazole, fenbuconazole, myclobutanil, propiconazole, tebuconazole, triadimefon, miconazole or fiucytosine acid salt.

12. The method as defined in claim 4 wherein the cation is an amine acid salt of a antibiotic composition.

13. The method as defined in claim 12 wherein the cation is tetracycline, clindamycin, tazarotene erythromycin, clinafloxacin, doxycycline, minocycline or lincomycin acid salts.

14. The method as defined in claim 1 wherein the anion is a phenolic, hydroxyl carboxylic, beta keto carboxylic, carboxylic or sulfonamide, or combinations thereof.

15. The method as defined in claim 14 wherein the anion is a phenolic consisting of tricloson, hexyresorcinal or thymol.

16. The method as defined in claim 14 wherein the anion is a hydroxy carboxylic consisting of lactic, glycolic, gluconic, glyceric, or salicylic.

17. The method as defined in claim 4 wherein the anion is a carboxylic acid consisting of undecylenic, pantothenic, azelaic, retinoic acids, tretinoin, isotretinoin, or adapalene.

18. The method as defined in claim 2 wherein the acid is used as part of the dermatological complex.

19. The method as defined in claim 18 wherein the acid has a carboxylic functionality.

20. The method as defined in claim 19 wherein the carboxylic acid consists of salicylic, lactic, glyconic, gluconic, glyceric, azelaic, clinafloxacin, adapalene, pantothenic, retinoic, and undecylenic.

21. The method as defined in claim 2 wherein the base is used as part of the dermatological complexes.

22. The method as defined in claim 21 wherein the base is an amine containing compound capable of being protonated to form an amino acid salt complex.

23. The method as defined in claim 22 wherein the base consists of clotrimazole clindamycin, tebuconazole, chitosan, sulfacetamide, lincomycin, tazarotene, metronedazole, minocycline and doxycycline.

24. Dermatological complexes wherein the bioactive cation consist of azole antifingal compound having an amine acid salt functionality.

25. The complexes of claim 24 wherein the amine acid salt of the antifingal azoles are cloconazole, clotrimazole, cyproconazole, fenbuconazole, myclobutanil, propiconazole, tebuconazole, triadimefon, and miconazole.

26. Dermatological complexes wherein the bioactive cation consist of a antibiotic compound having a amine acid salt functionality.

27. The complexes of claim 26 wherein the amine acid salt of the antibiotic are tetracycline, clindamycin, tazarotene, erythromycin, clinafloxacin, doxycycline, minocycline or lincomycin.

28. Dermatological complexes wherein the anion consist of a carboxylate funcationality of the vitamin A metabolites collectively known as retinoic acids.
